(19) United States
(12) Patent Application Publication
(10) Pub. No.: US 2023/0089107 A1
(43) Pub. Date: Mar. 23, 2023

(54) INCUBATOR FOR ENRICHMENT DURING TRANSPORT

(71) Applicant: Institute for Environmental Health, Inc., Lake Forest Park, WA (US)

(72) Inventor: Mansour Samadpour, Lake Forest Park, WA (US)

(21) Appl. No.: 17/802,094

(22) PCT Filed: Feb. 19, 2021
(86) PCT No.: PCT/US2021/018833

§ 371 Date: Aug. 24, 2022

Related U.S. Application Data
(60) Provisional application No. 62/981,514, filed on Feb. 25, 2020.

(51) Int. Cl. C12M 1/00

(52) U.S. Cl. CPC C12M 23/52; C12M 23/34; C12M 41/14

(57) ABSTRACT

Provided are sealable transport incubators for uniform incubation of sample specimens (e.g., microbial samples) during transport between two locations (e.g., microbial sampling and detection locations, the incubators comprising a multi-slot thermal-transfer divider each in uniform thermal communication with a heat source (e.g., electrical or phase change materials (PCM), and configured to provide for uniform heat transfer and distribution to the sample specimen during transport in the transport incubator. Additionally provided are methods for expediting provision of microbial assay detection results (e.g., using the disclosed sealable transport incubators).

### FIELD OF THE INVENTION

Aspects of the invention relate generally to incubating and enriching microbes (e.g., bacteria, pathogens, indicator organisms, etc.) in a culture medium prior to testing/assaying for microbial presence (e.g., detection), and more particularly to incubation devices configured to provide for uniform heat transfer and distribution for sustained uniform enrichment culturing of the microbes during transport (e.g., vehicle, aircraft, or any other delivery service), with or without an external power source, to a receiving location for microbial detection (e.g., an assay laboratory, etc.). Additional incubator aspects relate to the use of heat sources and/or heat-storage material (e.g., phase-change materials, etc.) configured to be in thermal (e.g., heat-exchange/transfer) communication with a multi-slot, contact-thermal-transfer divider. Further aspects relate to methods, using the uniform heat-transfer incubation devices, for reducing the time required to obtain detection results upon receipt of the incubation devices at the receiving location.

### BACKGROUND

To comply with safety and regulatory requirements, test samples, taken from product lots) to be assayed for microbes (e.g., for microbial contaminants, pathogenic microbes, etc.), typically require enrichment culturing, preferably in the form of a controlled prior culturing of the sample in a suitable enrichment medium at a suitable sustained temperature(s) over a sufficient time period to expand the number of microbes to a uniform level, detectable by a specific approved detection assay. The enrichment procedure must be compliant with the regulatory requirements of the assay procedure to ensure reliability of the test results. The site of test sample gathering (e.g., sampling plant material in a field, or sampling meat in a meat fabrication/processing facility), however, may be remote from the microbial assay/detection site, requiring that the test sample be transported (e.g., in some form of a transport container) to the detection site. Typically, because of difficulties in maintaining and documenting a compliant sustained enrichment incubation environment in such transport scenarios/containers (which typically lack accessible power sources, and/or lack uniform heat distribution, and/or which may be accident/spill prone, etc.), enrichment steps/processes (e.g., 12-24 hours) required prior to microbial detection assay are initiated only after receipt of the transported sample at the detection site, thus substantially delaying release of final detection assay results, and thereby delaying validation of the sampled product lot(s) for release into commerce. Such ‘transport delays’ are costly across the chain of commerce due, for example, to the perishable nature of products requiring microbial testing, and the requirement for holding/storage of the sampled product lots in secured, suitably controlled, refrigerated storage facilities) prior to their validated release into commerce (i.e., after confirming that the respective test samples tested negative for microbial contamination under compliant assay/testing procedures).

### BRIEF DESCRIPTION OF THE DRAWINGS

FIG. 1 shows, by way of non-limiting examples of the present invention, a frontal upper perspective view of an open configuration of an exemplary embodiment of the present invention.

FIG. 2 shows, by way of non-limiting examples of the present invention, a frontal upper perspective view of a closed configuration of the exemplary embodiment of the present invention

FIG. 3 shows, by way of non-limiting examples of the present invention, a rear upper perspective view of an open configuration of the exemplary embodiment of the present invention.

FIG. 4 shows, by way of non-limiting examples of the present invention, an upper perspective view of the aluminum frame 104 as shown in FIG. 1.

FIG. 5 shows, by way of non-limiting examples of the present invention, a bottom perspective view of the aluminum frame 104 as shown in FIG. 1.

FIG. 6 shows, by way of non-limiting examples of the present invention, an exploded view showing construction of the aluminum frame 104.

FIG. 7 shows, by way of non-limiting examples of the present invention, an upper perspective view of the aluminum frame 104 with the outer covers removed and dividers inserted in both layers.

FIG. 8 shows, by way of non-limiting examples of the present invention, an exploded view of the aluminum frame 104 with the outer covers removed and dividers 203 and middle plate 212 removed.

FIG. 9 shows, by way of non-limiting examples of the present invention, an open assembled view without aluminum frame 104.

FIG. 10 shows, by way of non-limiting examples of the present invention, a deconstructed view of the invention without aluminum frame 104 and shipping vessel 101A/101B.

FIG. 11 shows, by way of non-limiting examples of the present invention, an exploded view of the assembly shown in FIG. 10.

FIG. 12 shows, by way of non-limiting examples of the present invention, a frontal view of electronics box 102.

FIG. 13 shows, by way of non-limiting examples of the present invention, an exploded view of the electronics box 102.

FIG. 14 shows, by way of non-limiting examples of the present invention, a frontal upper perspective view of an open configuration of an additional exemplary embodiment of the present invention.

FIG. 15A shows, by way of non-limiting examples of the present invention, a frontal upper perspective view of a closed configuration of the additional exemplary embodiment of the present invention.

FIG. 15B shows, by way of non-limiting examples of the present invention, a frontal upper perspective view of the basket 402A and dividers 402B of the additional exemplary embodiment.

FIG. 15C shows, by way of non-limiting examples of the present invention, an inner upper perspective view of Phase Change Material (PCM) as used in the additional embodiment of the present invention.

FIG. 16 shows, by way of non-limiting examples of the present invention, a frontal upper perspective view of an open configuration of the additional exemplary embodiment of the present invention.

FIG. 17 shows, by way of non-limiting examples of the present invention, a frontal upper perspective view of an open further exemplary embodiment of the present invention.

FIG. 18 shows, by way of non-limiting examples of the present invention, a frontal upper perspective view of a closed configuration of the further exemplary embodiment of the present invention.

FIG. 19 shows, by way of non-limiting examples of the present invention, a frontal upper perspective view of the basket and dividers of the further exemplary embodiment.

FIG. 20 shows, by way of non-limiting examples of the present invention, a frontal bottom perspective view of the basket of the further exemplary embodiment.

FIG. 21 shows, by way of non-limiting examples of the present invention, a frontal upper perspective view of the heating device of the further exemplary embodiment.

FIG. 22 shows, by way of non-limiting examples of the present invention, a frontal bottom perspective view of the heating device of the further exemplary embodiment.

### SUMMARY OF EXEMPLARY EMBODIMENTS OF THE INVENTION

Embodiments of the disclosure can be described in view of the following clauses:

1. A transport incubator for uniform incubation of sample specimen during transport, comprising: a heat source; a multi-slot contact-thermal-transfer divider having a plurality of sample specimen incubation slots or compartments each slot or compartment having one or more internal surfaces in uniform thermal communication with the heat source, and configured, in operation of the device, to provide sufficient direct contact between the one or more internal surfaces of the slot or compartment and a surface or surfaces of a sample specimen container insertable therein to provide for uniform heat transfer and distribution to the sample specimen during transport in the transport incubator; and

a sealable housing configured to sealably house the heat source and the multi-slot divider within the housing during transport.

2. The transport incubator of clause 1, further comprising a temperature and/or time recording and/or verification device in communication with multi-slot contact-thermal-transfer divider, and configured for recording and/or verifying a thermal incubation history during transport and/or residence time of a sample specimen insertable within the multi-slot contact-thermal-transfer divider of the transport incubator.

3. The transport incubator of clause 1 or 2, wherein the multi-slot contact-thermal-transfer divider is configured to have two or more multi-slot tiers or layers, each tier comprising a multi-slot contact-thermal-transfer divider in uniform thermal communication with heat source.

4. The transport incubator of any one of clauses 1-3, wherein the multi-slot contact-thermal-transfer divider is housed within a frame.

5. The transport incubator of any one of clauses 1-4, wherein the multi-slot contact-thermal-transfer divider and/or the frame is reversibly removable from the transport incubator.

6. The transport incubator of any one of clauses 1-5, wherein the heat source comprises a phase change material (PCM) in uniform thermal communication with the multi-slot contact-thermal-transfer divider.

7. The transport incubator of clause 6, wherein the phase change material (PCM) is configured in a plurality PCM portions or panels, each in thermal contact with the multi-slot contact-thermal-transfer divider.

8. The transport incubator of clause 7, wherein the sealable housing comprises a reversibly sealable lid with an inner lid surface having one or more of the PCM portions or panels attached thereto, and wherein the plurality of PCM portions or panels are configured, upon sealing of the lid, to be in thermal contact with the multi-slot contact-thermal-transfer divider on multiple, or all sides thereof.

9. The transport incubator of any one of clauses 1-8, wherein the multi-slot contact-thermal-transfer divider and/or the frame comprises, or is formed of metal.

10. The transport incubator of any one of clauses 1-9, wherein the multi-slot contact-thermal-transfer divider and/or the frame comprises, or is formed of aluminum.

11. The transport incubator of any one of clauses 1-9, wherein each slot or compartment of the contact-thermal-transfer divider comprises at least two opposed internal surfaces configured to sandwich, by direct surface contact, surfaces of a sample specimen container insertable therebetween.

12. A method of for expediting provision of microbial assay detection results, comprising: placing, at a first location, one or more microbial sample containers comprising enrichment medium in corresponding slots or compartments of a transport incubator configured to provide for uniform heat transfer and distribution at one or more specified enrichment temperature(s) for sustained uniform enrichment culturing of the one or more microbial sample containers during transport; transporting the one or more microbial sample containers in the transport incubator, and enrichment culturing of the one or more microbial sample containers during transport; receiving, at a second location, the one or more enriched microbial sample containers; verifying that the enrichment culturing during transport was as specified; and performing one or more microbial detection assays on the enriched cultures, wherein the enrichment culturing during transport reduces or eliminates the need for further enrichment culturing at the second site prior to performing the one or more microbial detection assays.

13. The method of clause 12, wherein the enrichment culturing during transport is over a sufficient time period to expand the number of microbes to a uniform level, detectable by a specific approved detection assay without the need for further enrichment culturing at the second site prior to performing the one or more microbial detection assays.

14. The method of clause 12 or 13, wherein the transport incubator is according to any one of clauses 1-10.

15. The method of any one of clauses 12-14, further comprising provision of the detection assay results to provide for expedited release of product lots corresponding to the microbial samples.

### DETAILED DESCRIPTION OF EXEMPLARY EMBODIMENTS

Particular aspects provide devices (e.g., transport incubators) and methods for reducing the overall time for product specimen testing in transport scenarios involving testing facilities/sites that are remote from the product sampling and/or product storage sites. The devices and methods enable substantially faster communication of microbial testing results to producers, reducing the pre-validation product ‘holding’ period, and in turn reducing costs across the chain of commerce. For example, the disclosed transport incubators provide a temperature insulated environment for maintaining enrichment temperature(s) for, e.g., 12 to 24 hours. The transport incubators (shipping vessels) are selected to meet enrichment time specifications.

Provided are transportable incubation devices having a heat source in thermal communication (heat-transfer communication) with a multi-slot (e.g., multiple contact slots, cells, wells, cavities, etc.), contact-thermal-transfer divider. Each contact slot of the divider is configured to hold and contact a packaged (e.g., in a sample container) sample, during transport within the transport incubator. The multi-slot divider provides for uniform thermal communication between the heat source and surface(s) of the sample containers (and hence to the samples) within each contact slot of the multi-slot divider, to provides for uniform heat transfer and distribution to the sample during transport in the transport incubator. The heat source is in form of heat storage device or heating device connected to an electric source. The transportable incubation devices provide sustained, uniform heat-transfer to, and temperature maintenance of microbial sample enrichment cultures in contact with a contact slot of the divider, thereby enabling controlled, regulatory-compliant microbial enrichment culture of the samples during transport (e.g., to a testing laboratory).

In particular aspects, the multi-slot, contact-thermal-transfer divider (at times referred to as “multi-slot divider”) of the incubator comprises a metal (e.g., sheet metal) multi-slot divider, having slots (e.g., slots, cells, wells, cavities, etc.) configured to provide for direct contact between slot surfaces and surfaces of the enrichment sample container, to provide uniform heat transfer and distribution throughout all the enrichment samples inserted into the slots. The multi-slot divider may be made out of, or comprise, aluminum due to its light weight and fast heat conductivity characteristics. Preferably, the divider sandwiches the media sufficiently tightly to make direct contact between metal surface and sample container/package material to optimize heat transfer between the sample specimen media and the multi-slot divider. Preferably, one sample specimen container is inserted into each slot, as double- or multiple-loading of sample containers into a single slot may impair heat transfer between the multi-slot divider and the media of the sample specimens. In particular aspects, therefore, a double stack (e.g., double-deck) multi-slot, contact-thermal-transfer divider is provided, for doubling the number of slots, so that more sample specimens may be uniformly incubated without double-loading sample specimens into a single slot of the multi-slot divider. Particular double stack (double-deck) multi-slot divider aspects comprise a middle insert divider to divide the basket into two rows. The middle insert is removable to access the bottom layer of slots.

In aspects of the invention, test samples potentially containing a target microbe (e.g., such as Listeria monocytogenes, Salmonella, E. coli, and/or other microbial pathogens, or other microbe, etc.), and comprising an enrichment medium suitable to provide for enrichment of the target microbe are packaged in a sample container and loaded into a contact slot of the multi-slot, contact-thermal-transfer divider of the incubator, which may be pre-set to provide and maintain an optimal and uniform temperature for controlled, compliant enrichment of the microbial target. Preferably, the sample container is of a suitable material and/or is suitably configured to contact and conform to surfaces (e.g., at least one, at least two, at least three, at least four, at least five, etc.) of the slot of the multi-slot, contact-thermal-transfer divider, such that the packaged samples receive uniform heat-transfer and distribution throughout each and every sample being transported in the transport incubator, to ensure compliant enrichment and avoid faulty test results.

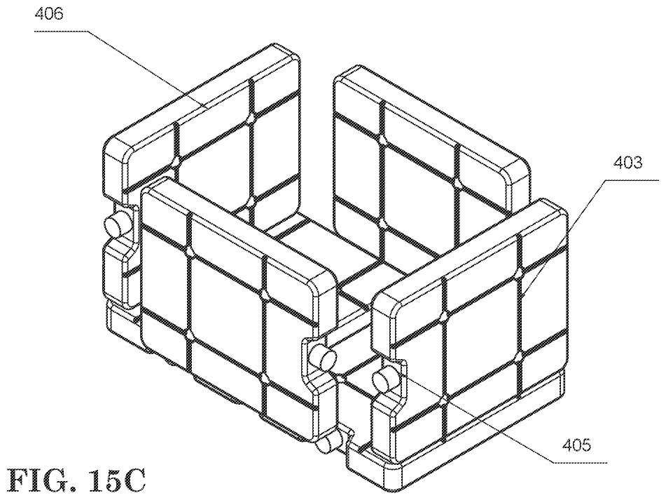

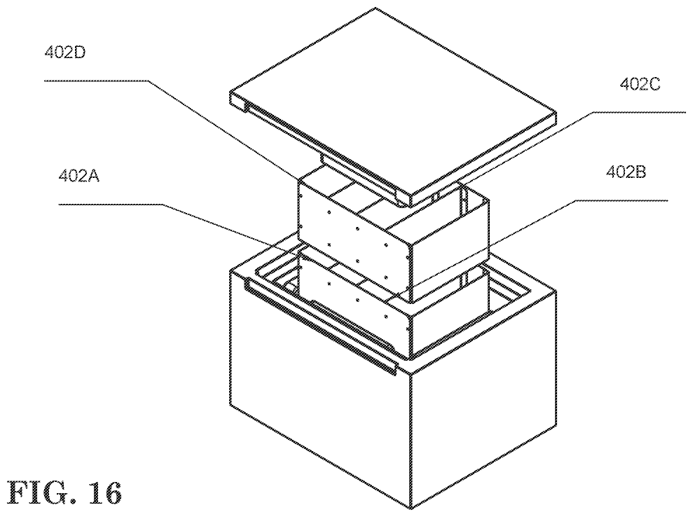

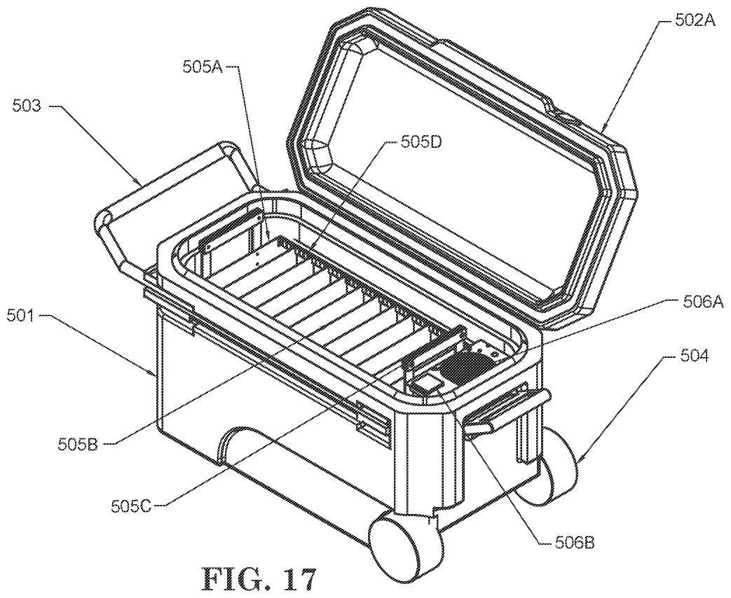

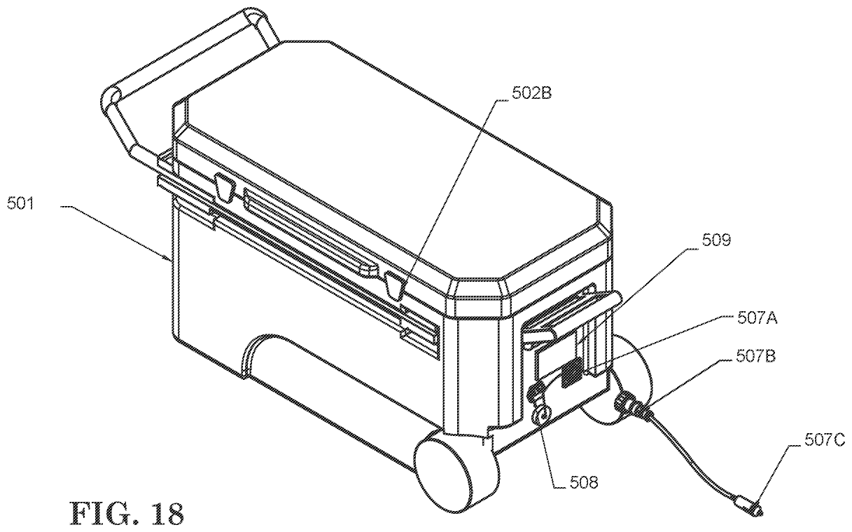

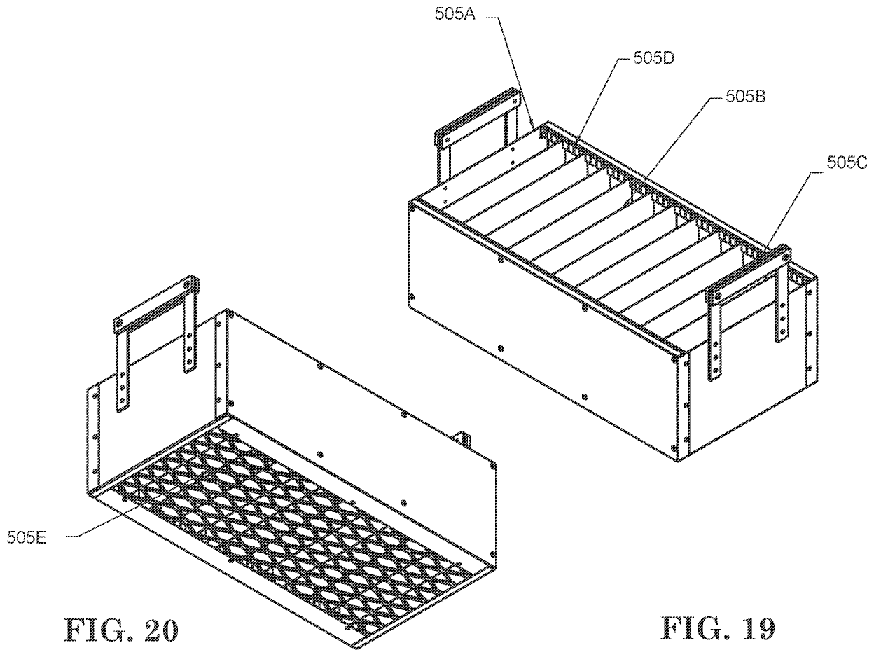

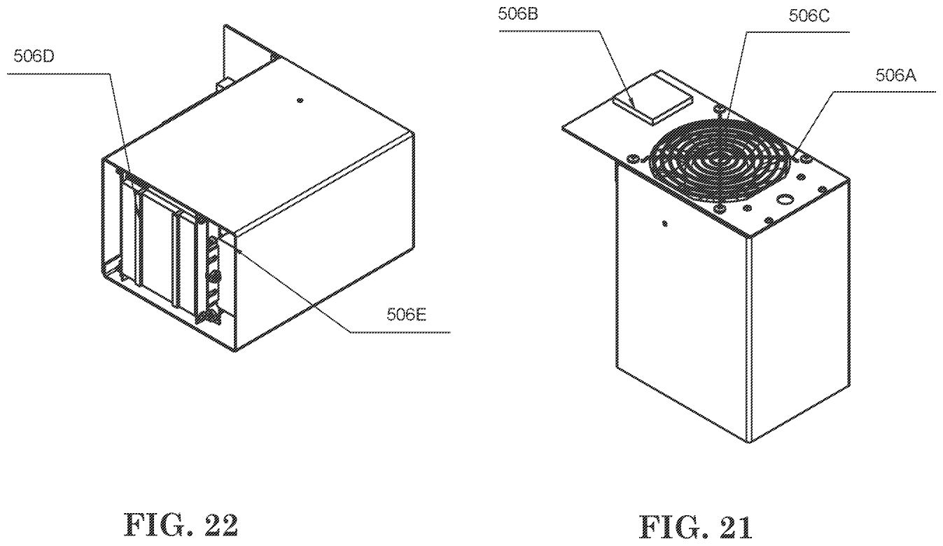

In an exemplary first embodiment (and with reference to FIGS. 17-22), enriching sample specimens during transport in a transport incubator/vessel is provided by charging (e.g., using an external power source of a vehicle), and maintaining temperature(s) using a multi-slot, contact-thermal-transfer divider in thermal communication (heat-transfer communication) with a heat source. For example, vehicle electricity may be used to power up a heater fan to maintain the incubator at one or more enrichment temperature(s) during transporting. Vehicle battery life may be affected, as the heater fan may draw large amounts of power. Moreover, such transport incubator embodiments are not completely portable, because of the need for electricity or other external power source.

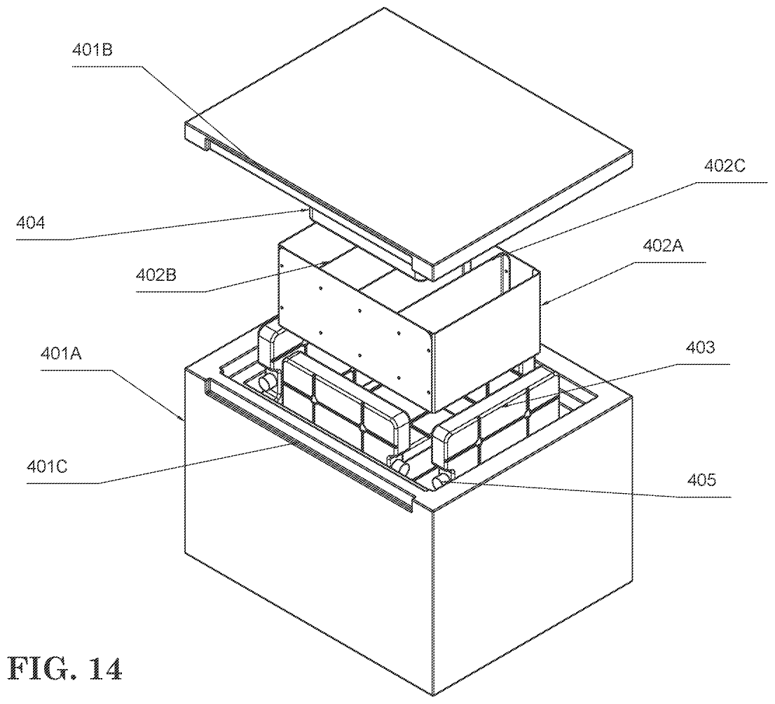

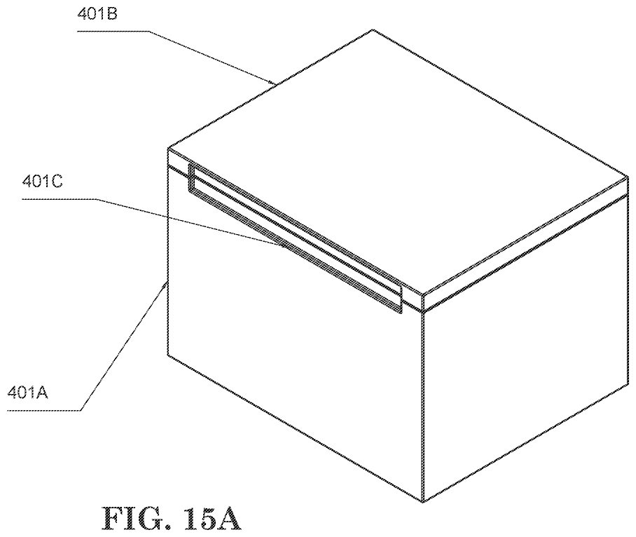

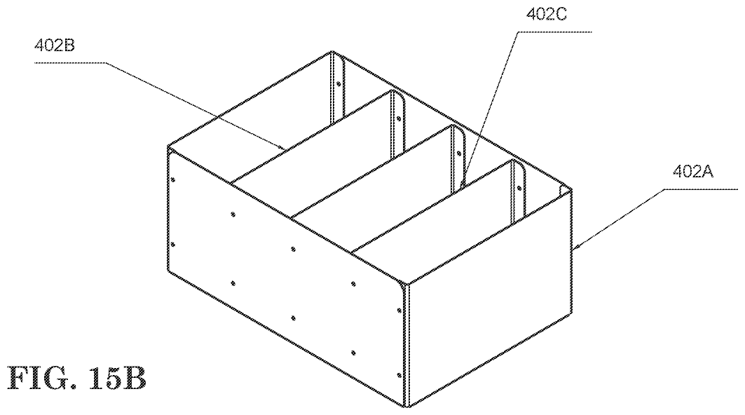

In an exemplary second embodiment (and with reference to FIGS. 14-16), potential vehicle battery power and portability limitations are overcome by enriching sample specimens during transport in a transport incubator/vessel using an incubator (e.g., a powerless incubator) having one or more heat-storage device(s) (e.g., integral or reversibly insertable heat-storage devices). For example, one or more integral or insertable heat-storage device(s) of a transport incubator may be pre-charged (e.g., prior to transport) with external power source, to maintain temperature during transport of sample specimens in contact with contact slots of the multi-slot, contact-thermal-transfer divider, which is in thermal communication (heat-transfer communication) with the heat-storage device(s) in the incubator. The heat-storage device(s) may comprise one or more phase change material(s) (PCM) to store and provide/transfer heat prior to and/or during transportation. For example, latent heat, associated with phase changes such as freezing and melting of phase change materials, may be used to store energy and release the stored energy to surroundings, based on phase changes. For example, melting of a PCM may draw-in/store a substantial amount of energy or heat to complete a melting stage, and freezing/solidification of the melted/liquid state material may then release the energy or heat to the surroundings. In embodiments of the invention, PCM heat-storage materials are in thermal communication (heat-transfer communication) with the multi-slot, contact-thermal-transfer divider, which in turn is in contact-thermal communication with the sample specimens being incubated. In this way, the energy and heat released by the freezing phase of the phase change material may uniformly supply the heat and maintain uniform enrichment temperature(s) in the transport incubator without an external power source. In particular aspects of such PCM embodiments, PCM may be melted within the shipping vessel and then used to maintain enrichment temperature prior to transporting the incubator vessel. For example, the heating device (e.g., comprising one or more PCM units/devices) is incorporated in a basket (e.g., of the incubator), and prior to transportation the transport incubator (shipping vessel) is plugged into any residential or commercial power source to preheat the PCM(s). The PCM(s) are structured so that heat can be distributed to each PCM device(s) in the shipping vessel. Prior to loading samples specimens, the shipping vessel may be melting PCM at higher temperature than the enrichment temperature to speed up the melting process. After the melting cycle, the shipping vessel and/or the multi-slot, contact-thermal-transfer divider, which in turn is in thermal communication between the PCM(s), is cooled down to the desired enrichment temperature which is maintained throughout loading of the specimen samples into the multi-slot, contact-thermal-transfer divider. In particular aspects, PCM comprising organic, inorganic or microencapsulated PCM is used (e.g., as available from Lizoo Commodity Co., Ltd), and may, for example, be structured as panels. PCMs may include, for example, plant-oil-based material. PCMs may comprise eutectics, salt hydrates, organic materials, and high temperature salts. Preferred PCMs are, or comprise, wax materials.

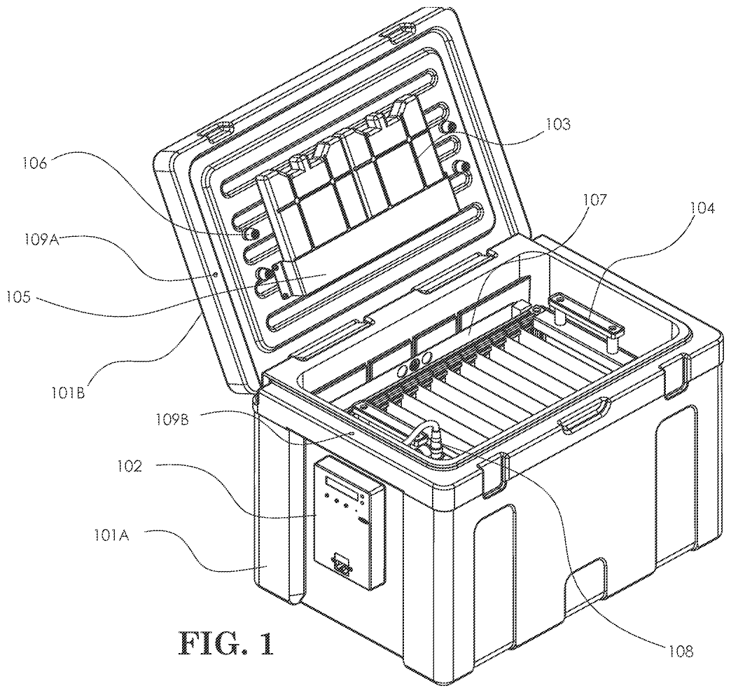

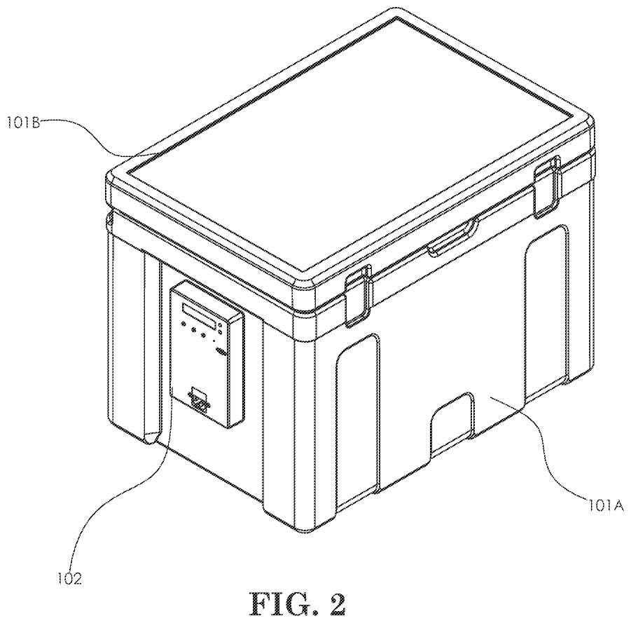

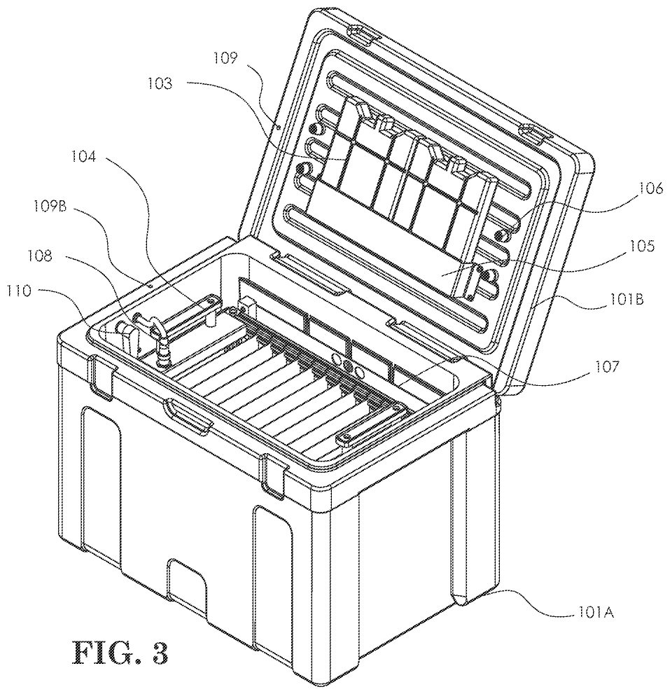

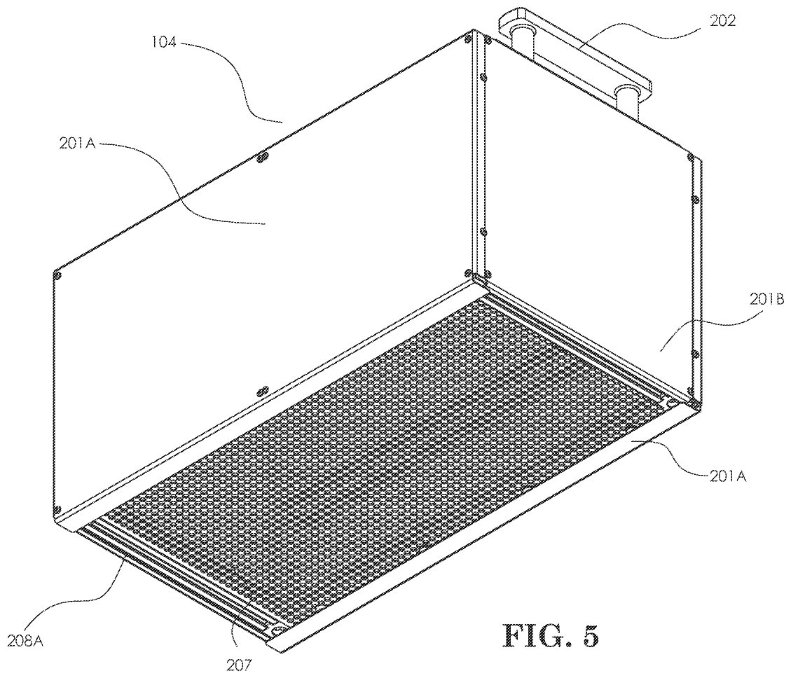

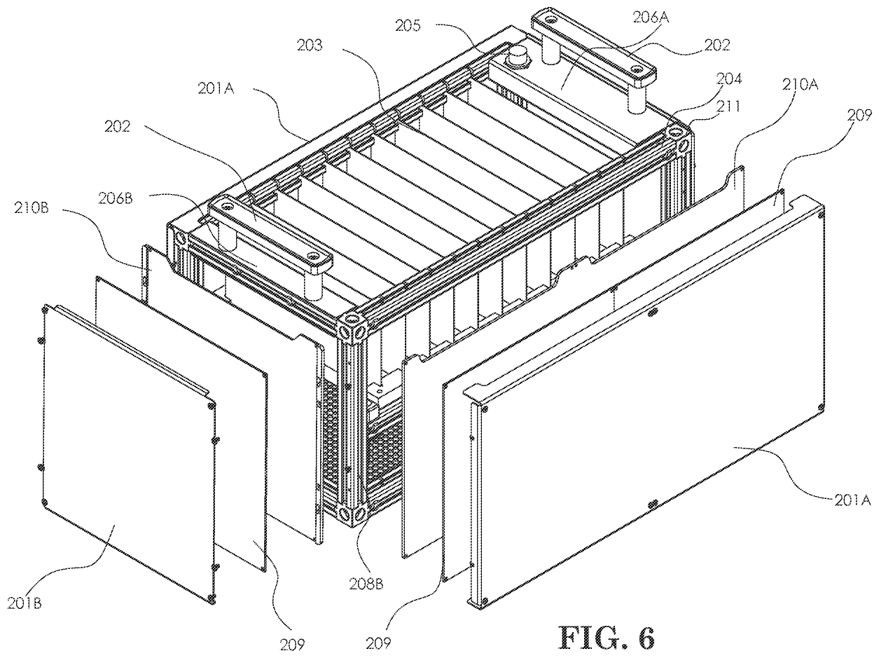

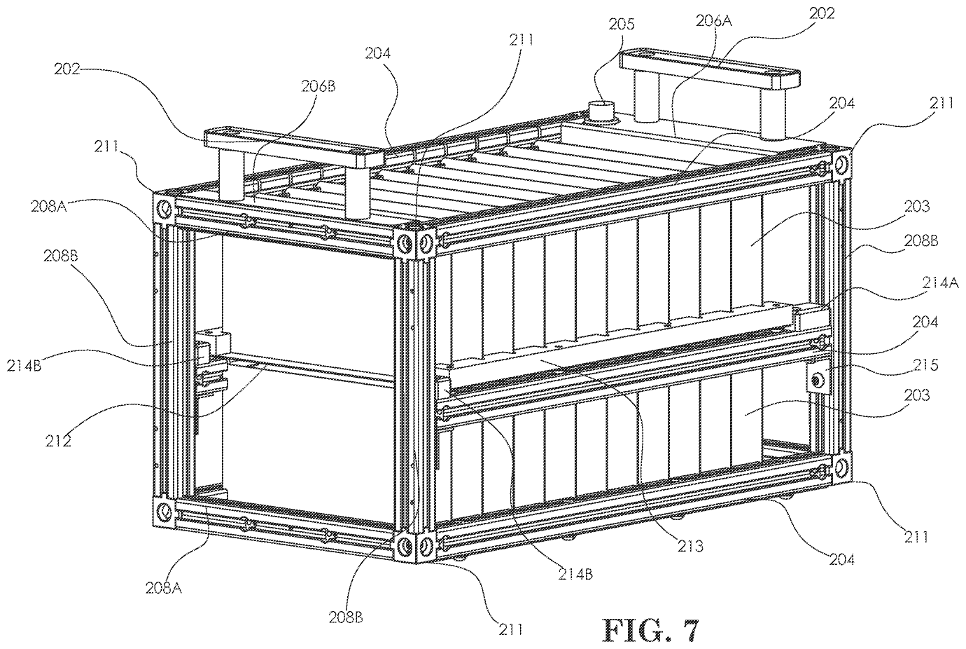

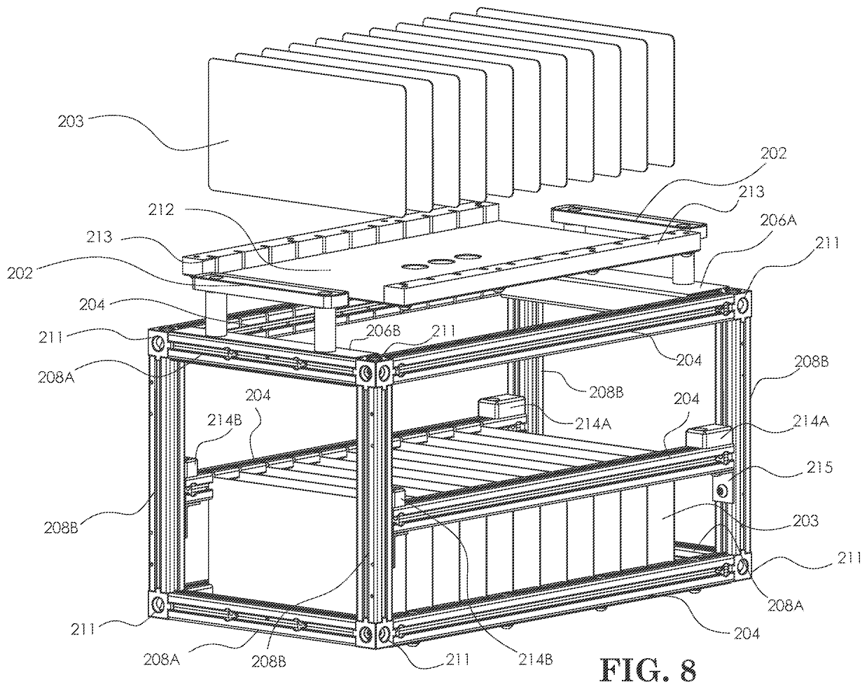

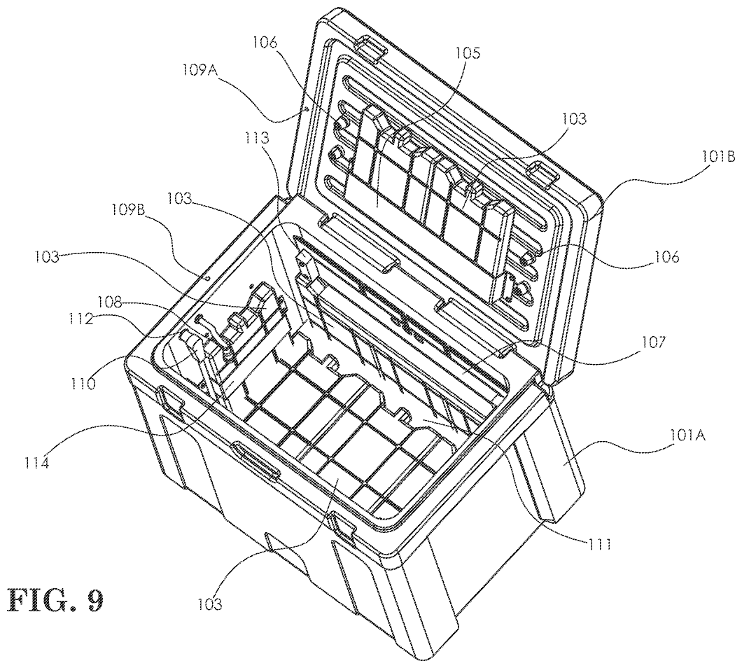

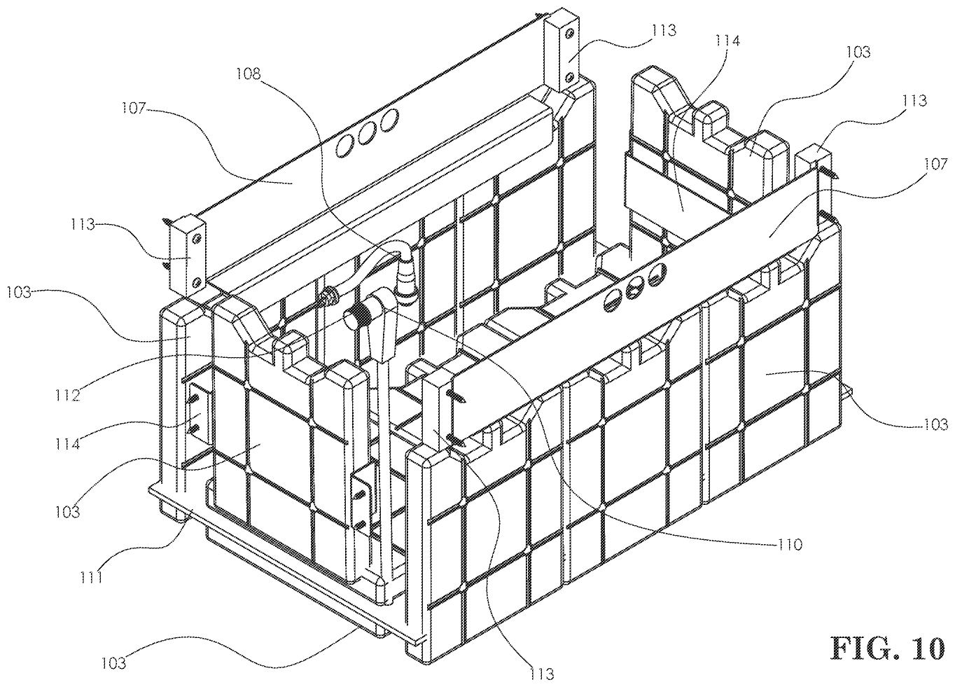

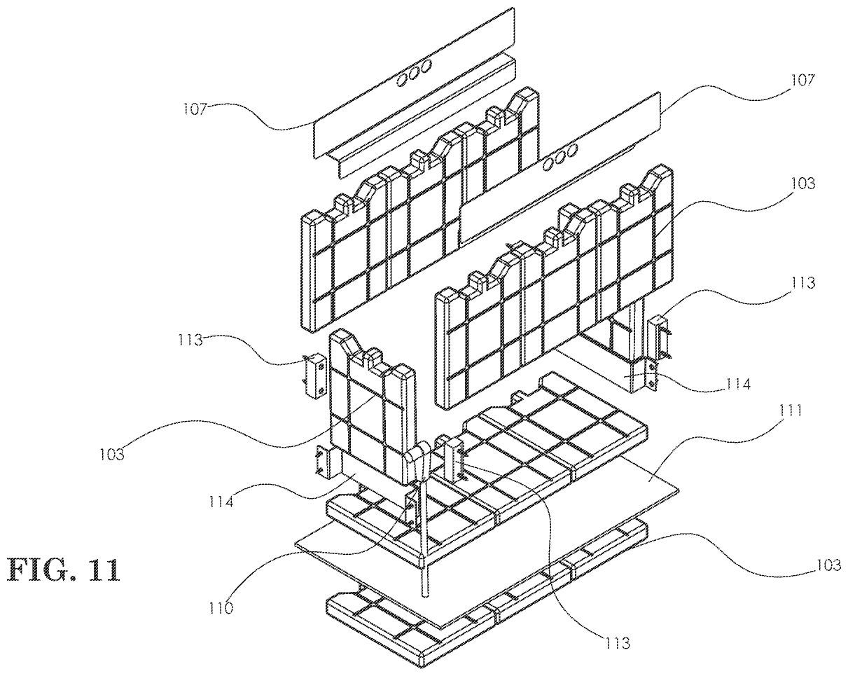

In an exemplary third embodiment (and with reference to FIGS. 1-13), a transport incubator having a power source (e.g., a built-in power source) and one or more heat-storage device(s) is provided. For example, a heat-storage device of a transport incubator is pre-charged using a built-in (e.g., internal) power source, the sample specimens are loaded into contact slots of a multi-slot [contact-thermal-transfer] divider within the incubator (e.g., while maintaining optimal temperature(s)), and the sample specimens are transported while maintaining optimal temperature(s) without the use or need of an external power source.

The transport incubators may have, and preferable do have a temperature/time verification device, used and recognized in the art for recording and verifying the enrichment incubation achieved during transport, including within the transport incubators disclosed herein.

In preferred embodiments of the transport incubators, the transport incubator is sealable, (e.g., fully sealable) to prevent media leakage to outside of transport incubator (shipping vessel).

Additional embodiments provide transport incubator embodiments as disclosed herein, for use in combination with gel media. Such gel media embodiments provide a solution to problems relating to leaking of liquid media during transport, which can lead to flooding within the transport incubator and contamination.

### EXEMPLARY WORKING EXAMPLES

The following working Examples are provided and disclosed for illustrative purposes to demonstrate exemplary embodiments of the invention for, and are not intended to limit the scope of the inventive devices, methods, and applications.

### Example 1

### A Transport Incubator Having a Power Source (e.g., a Built-In Power Source) and One or More Heat-Storage Device(s) Was Constructed

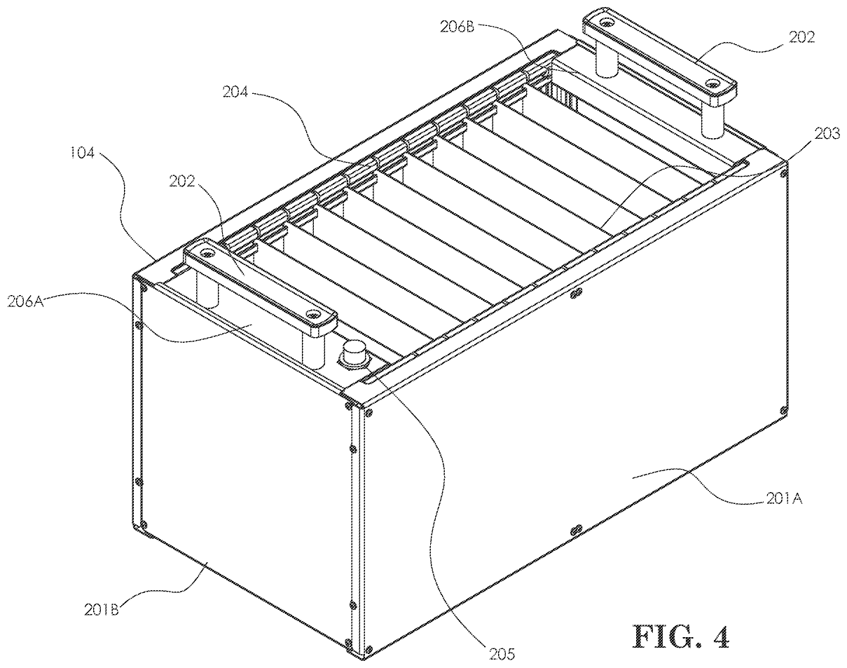

With reference to FIGS. 1-4 and 9-11, 101A is the bottom half of an insulated shipping vessel 100. 101B is the upper half of the insulated shipping vessel 100. An electronics control box 102 is attached to the bottom half 101A. Phase Change Material (PCM) 103 may be at any desired temperature. Aluminum frame 104 (multi-slot divider) holds samples (e.g., packaged sample specimens). Bent aluminum sheet metal 105 holds the PCM on 101B. Aluminum & rubber bumpers 106 hold the frame 104 in place when closed. Bent removable aluminum sheet 107 holds PCM down. Pigtail electrical connector 108 connects to 205 (see FIG. 4) on frame 104. Magnet 109A is for switching operation on/off. Magnetic switch 109B allows operation when in proximity to 109A. Electrical connection 110 is attached to silicon heating pad 111 and connects to connection 112 on 101A. Silicone heating pad 111 heats up the PCM 103. Electrical connector 112 is attached (e.g. glued) to 101B that connects to 110. Plastic rail 113 holds 107 in place. Bent aluminum sheet metal 114 that holds the PCM 103 in place.

With reference to FIGS. 4-8, aluminum sheet metal cover 201A provides the sides of the frame 104. Aluminum sheet metal 201B provides the frame 104 ends. Handle 202 is for carrying the frame 104. Removable sheet metal divider 203 defines slots within the frame 104. Slotted aluminum extrusion 204 is used for frame construction. Electrical connection 205 provides for electrical connectivity. Horizontal aluminum sheet metal cover 206A provides cover for the electronics front end. Horizontal aluminum sheet metal cover 206B provides cover for the rear end. Aluminum mesh bottom 207 forms a bottom of frame 104. Horizontal aluminum extrusion 208A is also for frame 104 construction. Vertical aluminum extrusion 208B is also for frame 104 construction. PCB heatbed 209 facilitates heating (e.g., heats) the system up. Side plastic cover 210A protects the heatbeds 209. End plastic cover 210B protects the heatbeds 209. Aluminum corner pieces 211 are used for frame 104 construction. Middle insert 212 divides frame 104 into two rows. Black slotted plastic bars 213 are used to align divider 203. Long plastic stop 214A aligns middle insert 212. Short plastic stop 214B middle insert 212. Angled bracket 215 holds up extrusion 204 in the middle.

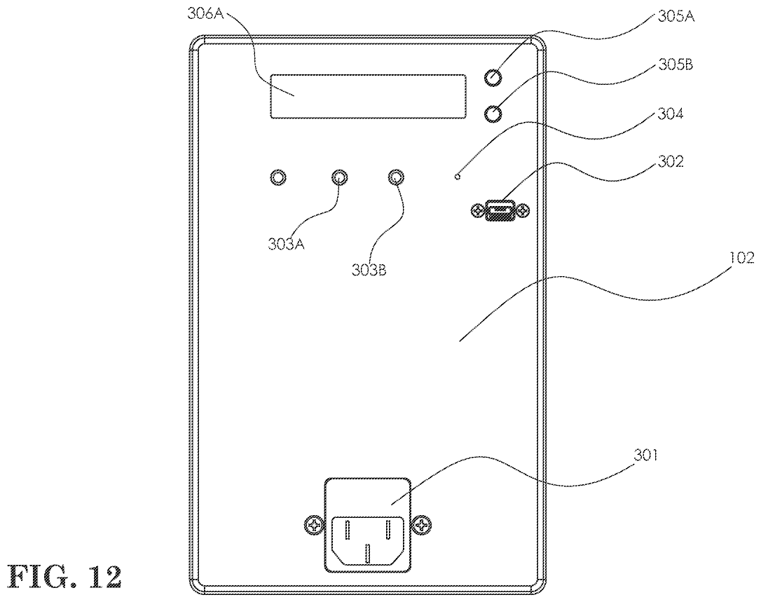

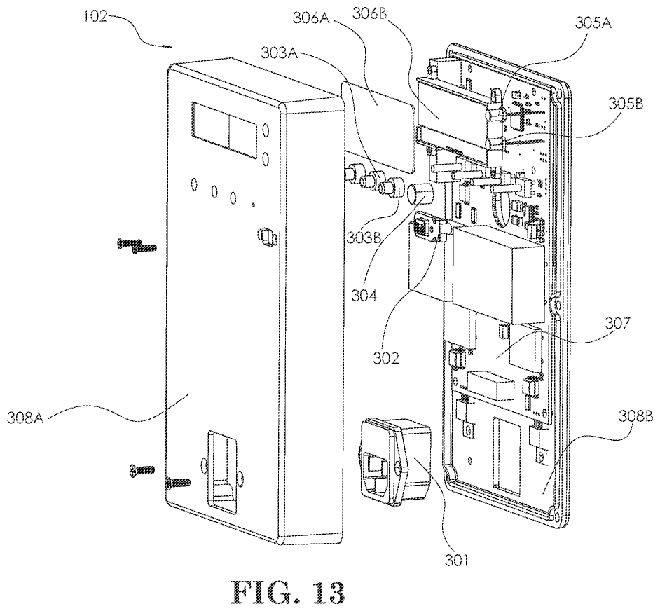

With reference to FIGS. 12 and 13, power inlet connector 301 connects to the wall outlet. Micro USB connector 302 allows for data extraction. Button 303A is for selecting YES or temperature setting (e.g., 37° C.). Button 303B for selecting NO or temperature setting (e.g., 42° C.). Button 304 is a reset button. LED 305A blinks or remains solid during the melting and heating phases of the PCM. LED 305B is of a different color than 305A, and that remains solid during incubation and shipping phases. Plastic cover 306A is for the LCD screen 306B. LCD screen 306B displays texts and performance options. Programmable PCB board 307t controls the entire system. Main body 308A of the shell of the electronics box 102. Lid plate 308B is the lid of the shell of the electronics box 102.

### Example 2

### A Transport Incubator/Vessel Using an Incubator (e.g., a Powerless Incubator) Having One or More Heat-Storage Device(s) (e.g., Integral or Reversibly Insertable PCM-Type Heat-Storage Devices) Was Constructed

With reference to FIGS. 14-16, 401A is a temperature insulated box. Lid 401B is a lid for the temperature insulated box 401A. Body 401C is the body of the temperature insulated box 401A. Basket 402A contains packaged samples. Divider 402B is used to make multiple compartments. Attachment of 402B to 402A is shown at 402C. Second or multiple basket 402D is stacked on top of first basket. Phase Change Material (PCM) 403 is used to provide any desired temperature. PCM 404 is PCM 403 surrounding samples in 402A. Cap and drain 405 for PCM 403. PCM 406 is PCM 403 surrounding samples in 402A.

### Example 3

### A Transport Incubator/Vessel Was Provided by Charging (e.g., Using an External Power Source of a Vehicle), and Maintaining Temperature(s) Using a Multi-Slot, Contact-Thermal-Transfer Divider in Thermal Communication (Heat-Transfer Communication) with a Heat Source

With reference to FIGS. 17-22, body 501 forms the body of an insulated box 500. Lid 502A provides a lid for insulated box 500. Latch 502B for lid 502A. Handle 503 on body 501. Wheel 504 on body 501. Basket 505A contains packaged samples. Multi-sample divider 505B is used to make multiple compartments. Handle 505C for basket 505A. Plastic rail 505D holds divider 505B in place. Aluminum mesh bottom 505E provides a bottom for basket 505A. Heating device 506A provides a heat source. Display and controller 506B for heating device 506A. Fan 506C in heating device 506A. Heating element 506D of heating device 506A. Control relay 506E of heating device 506A. Exemplary power input 507A for insulated box 500 (or other embodiments). Cable and connector 507B used to connect 507A from power source (not shown). Connector 507C is used to connect to the power source (not shown). Secondary power input 508 to wall power. Display and user interface 509 is provided for a user interface. There is a space between aluminum mesh bottom 505E and the internal bottom of body 501, providing an air channel to uniformly distribute heated air driven by fan 506C onto and into the slots of the multi-sample divider 505B.

1. A transport incubator for uniform incubation of sample specimens during transport, comprising:
a heat source;
a multi-slot contact-thermal-transfer divider having a plurality of sample specimen incubation slots or compartments each slot or compartment having one or more internal surfaces in uniform thermal communication with the heat source, and configured, in operation of the device, to provide sufficient direct contact between the one or more internal surfaces of the slot or compartment and a surface or surfaces of a sample specimen container insertable therein to provide for uniform heat transfer and distribution to the sample specimen dud ng transport in the transport incubator; and
a sealable housing configured to sealably house the heat source and the multi-slot divider within the housing during transport.

2. The transport incubator of claim 1, further comprising a temperature and/or time recording and/or verification device in communication with the multi-slot contact-thermal-transfer divider, and configured for recording and/or verifying a thermal incubation history during transport and/or residence time of a sample specimen insertable within the multi-slot contact-thermal-transfer divider of the transport incubator.

3. The transport incubator of claim 1, wherein the multi-slot contact-thermal-transfer divider is configured to have two or more multi-slot tiers or layers, each tier comprising a multi-slot contact-thermal-transfer divider in uniform thermal communication with heat source.

4. The transport incubator of claim 1, wherein the multi-slot contact-thermal-transfer divider is housed within a frame.

5. The transport incubator of claim 1, wherein the multi-slot contact-thermal-transfer divider and/or the frame is reversibly removable from the transport incubator.

6. The transport incubator of claim 1, wherein the heat source comprises a phase change material (PCM) in uniform thermal communication with the multi-slot contact-thermal-transfer divider.

7. The transport incubator of claim 6, wherein the phase change material (PCM) is configured in a plurality PCM portions or panels, each in thermal contact with the multi-slot contact-thermal-transfer divider.

8. The transport incubator of claim 7, wherein the sealable housing comprises a reversibly sealable lid with an inner lid surface having one or more of the plurality of PCM portions or panels attached thereto, and wherein the plurality of PCM portions or panels are configured, upon sealing of the lid, to be in thermal contact with the multi-slot contact-thermal-transfer divider on multiple, or all sides thereof.

9. The transport incubator of claim 1, wherein the multi-slot contact-thermal-transfer divider and/or the frame comprises, or is formed of metal.

10. The transport incubator of claim 1, wherein the multi-slot contact-thermal-transfer divider and/or the frame comprises, or is formed of aluminum.

11. The transport incubator of claim 1. wherein each slot or compartment of the contact-thermal-transfer divider comprises at least two opposed internal surfaces configured to sandwich, by direct surface contact, surfaces of a sample specimen container insertable therebetween.

12. A method of for expediting provision of microbial assay detection results, comprising:
placing, at a first location, one or more microbial sample containers comprising enrichment medium in corresponding slots or compartments of a transport incubator configured to provide for uniform heat transfer and distribution at one or more specified enrichment temperature(s) for sustained uniform enrichment culturing of the one or more microbial sample containers during transport;
transporting the one or more microbial sample containers in the transport incubator, and enrichment culturing of the one or more microbial sample containers during transport;
receiving, at a second location, the one or more enriched microbial sample containers;
verifying that the enrichment culturing during transport was as specified; and
performing one or more microbial detection assays on the enriched cultures, wherein the enrichment culturing; during transport reduces or eliminates the need for further enrichment culturing at the second site prior to performing the one or more microbial detection assays.

13. The method of claim 12, wherein the enrichment culturing during transport is over a sufficient time period to expand the number of microbes to a uniform level, detectable by a specific approved detection assay without the need for further enrichment culturing at the second site prior to performing the one or more microbial detection assays.

14. The method of claim 12, wherein the transport incubator is according to claim 1.

15. The method of claim 12, further comprising provision of the detection assay results to provide for expedited release of product lots corresponding to the microbial samples.
